(19) United States
(12) Patent Application Publication
(10) Pub. No.: US 2011/0264068 A1
(43) Pub. Date: Oct. 27, 2011

(54) DISPOSABLE BODILY FLUID HANDLING COMPOSITE PAD

(75) Inventor: Ayami Suga, Kagawa (JP)

(73) Assignee: UNI-CHARM CORPORATION, SHIKOKUCHUO-SHI, EHIME (JP)

(21) Appl. No.: 12/525,036

(22) PCT Filed: Jul. 28, 2009
(86) PCT No.: PCT/JP2009/063443

§ 371 Date: May 23, 2011

(51) Int. Cl. A61F 13/505

(52) U.S. Cl. 604/385.14

(57) ABSTRACT

The present invention aims to provide a disposable bodily fluid handling composite pad allowing the wearer to recognize what number component pad is being used and how many useful component pads remain. A menstruation napkin 1 as a typical example of the bodily fluid handling composite pad comprises a first component pad 10, a second component pad 20 and a third component pad 30 laminated one on another. On the first component pad 10, two groups each consisting of three butterflies are drawn so as to be diagonally opposed to each other as identifying element 61. On the second component pad 20, two groups each consisting of two butterflies, i.e., reduced by one from each of the groups on the first component pad 10, are drawn as identifying element 62. On the third component pad 30, the total number of butterflies is further reduced to a single pair which is drawn as identifying element 63.

### RELATED APPLICATIONS

The present application is based on, and claims priority from, International Application PCT/JP2009/63443, filed Jul. 28, 2009.

### TECHNICAL FIELD

The present invention generally relates to a disposable bodily fluid handling composite pad and more particularly to such a composite pad used in the form of, for example, a menstruation napkin, a urine absorbent pad or panty liner.

### RELATED ART

It is known to laminate two or more component pads one upon another and thereby to obtain a disposable bodily fluid handling composite pad. For example, PATENT CITATION 1 discloses such composite pad comprising laminated two or more liquid-absorbent component pads temporarily joined together along transversely opposite side edges by locally crimping these laminated component pads. It is possible for the wearer of such composite pad to peel off the component pad facing the wearer's body from the composite pad one by one. More specifically, when the component pad contacting the wearer's skin is getting soiled, it may be peeled off from the remaining components to expose the adjacent fresh component pad for use.

### PRIOR ART DOCUMENT

### Patent Citation

Patent Citation 1: JP 4226086 B2

### SUMMARY OF THE INVENTION

### Problem to be Solved by the Invention

In the course of using the composite pad comprising two or more component pads laminated one on another, it becomes sometimes unclear for the wearer what number component pad is being used. When the last component pad is used and soiled, the composite pad must be exchanged with a fresh composite pad. However, if the wearer is unaware of the fact that the component pad being used is the last component pad, the wearer might miss to prepare a fresh composite pad and to exchange the used composite pad with the fresh one. Furthermore, the wearer sometimes delude herself as if the last component pad has been soiled and exchanges the composite pad still containing one or more useful component pad with the fresh composite pad. As a consequence, the remaining composite pad or pads might be vainly thrown away.

In view of the problem as has been described above, it is an object of the present invention to provide a disposable bodily fluid handling composite pad allowing the wearer to recognize how many component pads remain.

### Measure to Solve the Problem

The present invention relates to an improvement of a disposable bodily fluid handling composite pad having a longitudinal direction, a transverse direction, an upper side facing a wearer's body and a lower side opposite to the upper side, and comprising component pads each including an upper liquid-absorbent sheet and a lower liquid-impervious sheet wherein two or more of the component pads are laminated one on another.

The disposable bodily fluid handling composite pad according to the present invention is characterized in that:

the two or more component pads respectively include temporarily joined regions along which the component pads are temporarily joined so as to be peeled off from the other component pads and identifying elements adapted to be visually recognized from the upper side; and

the identifying element displayed on one component pad is different from the identifying elements displayed on other component pads.

According to one preferred embodiment of the present invention, the identifying element includes at least one of letter, graphic and symbol as a constituent.

According to another preferred embodiment of the present invention, the identifying element includes a numeric conception as the constituent.

According to another preferred embodiment of the present invention, the identifying element includes the constituent having a series of conception and displayed depending on order in which the component pads laminated one on another.

According to another preferred embodiment of the present invention, the identifying element comprises the same number of the letter or graphic or symbol as the number of the component pads counted from the lower side.

According to another preferred embodiment of the present invention, the identifying element comprises two or more of the graphics or symbols having a conception of temporal change and the graphics or symbols are differently displayed on each of the component pads so as to display the conception of temporal change corresponding to the order of lamination of the component pads.

According to another preferred embodiment of the present invention, the temporarily joined regions are formed along a peripheral edge of the component pads except a partial segment of the peripheral edge by a heat crimping treatment so that the partial segment left free from an effect of the heat crimping treatment serves as a guide region to indicate an appropriate region to be pinched by the wearer's fingers when it is desired to be peeled off the soiled component pad from the composite pad.

### EFFECT OF THE INVENTION

Each of two or more liquid-absorbent component pads laminated one on another includes identifying element adapted to be visually recognized from the upper side and such identifying element on one component pad is different from the identifying element on the other component pad so that the individual component pads can be identified. The wearer can recognize what number component pad is being used and how many useful component pads remain on the basis of the particular pattern of the identifying element. In this way, the wearer can exchange the soiled composite pad with a fresh composite pad at the right time without wastefulness.

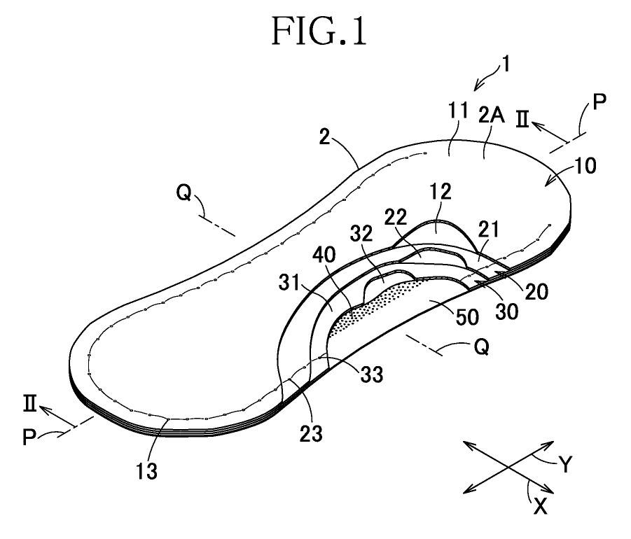

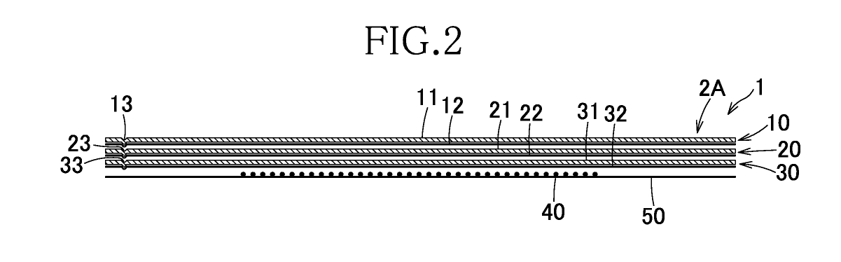

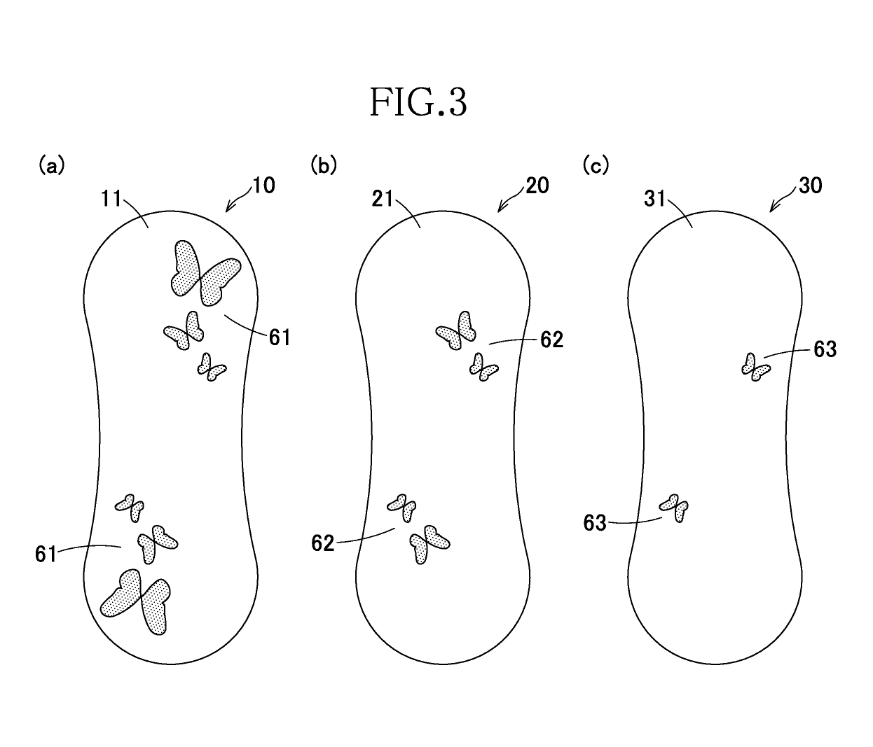

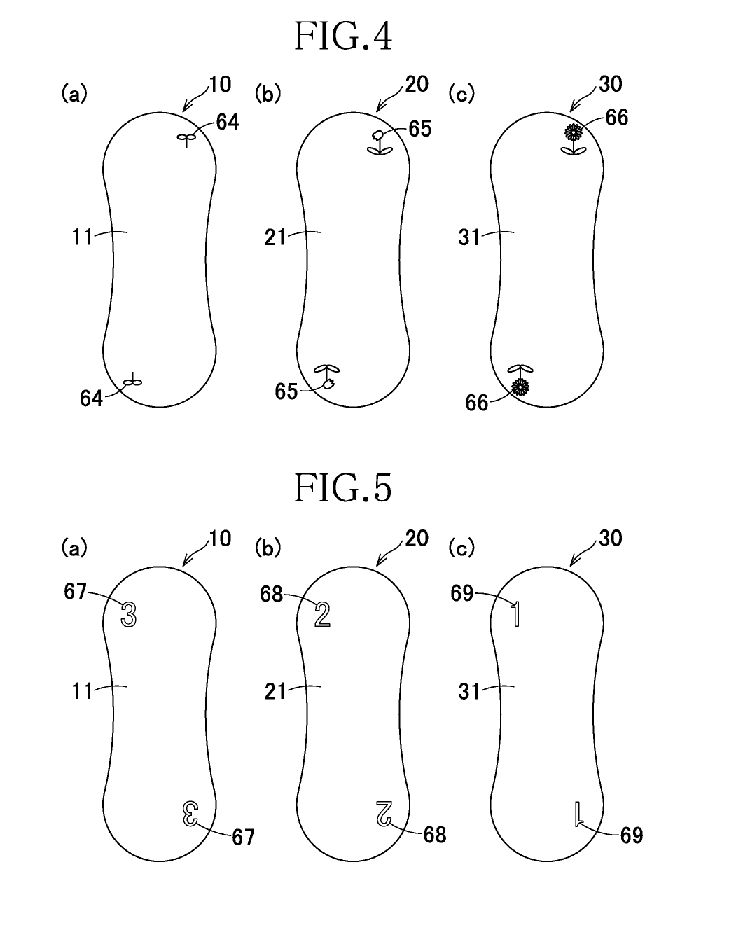

### BRIEF DESCRIPTION OF THE DRAWINGS

[FIG. 1] FIG. 1 is a perspective view of a menstruation napkin.

[FIG. 2] FIG. 2 is a sectional view taken along the line II-II in FIG. 1.

[FIG. 3] FIG. 3 is a schematic diagram illustrating Example 1.

[FIG. 4] FIG. 4 is a schematic diagram illustrating Example 2.

[FIG. 5] FIG. 5 is a schematic diagram illustrating Example 3.

### IDENTIFICATION OF REFERENCE NUMERALS USED IN THE DRAWINGS

1 menstruation napkin (disposable body fluid handling composite pad)
2A guide region
10 first pad (pad)
11 liquid-absorbent sheet
12 liquid-impervious sheet
13 temporarily joined region
20 second pad (pad)
21 liquid-absorbent sheet
22 liquid-impervious sheet
23 temporarily joined region
30 third pad (pad)
31 liquid-absorbent sheet
32 liquid-impervious sheet
33 temporarily joined region
61-69 indicator element
X transverse direction
Y longitudinal direction

### DETAILED DESCRIPTION FO THE PREFERRED EMBODIMENTS

Details of the disposable bodily fluid handling composite pad according to the present invention will be described hereunder on the basis of a menstruation napkin as one of typical embodiments of the invention. Construction of the menstruation napkin 1 shown by FIGS. 1 and 2 are common to Examples 1 through 3. FIG. 1 is a perspective view showing the menstruation napkin 1 as partially cutaway for convenience of illustration and FIG. 2 is a sectional view taken along the line II-II in FIG. 1. The menstruation napkin 1 has a longitudinal direction Y and a transverse direction X wherein the menstruation napkin 1 is relatively long in the longitudinal direction Y and has a width gradually reduced from longitudinally opposite ends toward a central region. The menstruation napkin 1 has the imaginary longitudinal center line P-P bisecting a width dimension in the transverse direction X and the imaginary transverse center line Q-Q bisecting a length dimension in the longitudinal direction Y. The menstruation napkin 1 is shaped substantially symmetric about the imaginary longitudinal center line P-P as well as about the imaginary transverse center line Q-Q.

The menstruation napkin 1 has an upper side facing the wearer's body and the opposite lower side facing the wearer's garment. The menstruation napkin 1 comprises two or more liquid-absorbent component pads laminated from the upper side to the lower side. Specifically, the menstruation napkin 1 comprises a first component pad 10 defining the uppermost layer of the laminate, a second component pad 20 underlying the first component pad 10 and a third component pad 30 underlying the second component pad 20. These first, second and third component pads 10, 20, 30 respectively comprise upper liquid-absorbent sheets 11, 21, 31 and lower liquid-impervious sheets 12, 22, 32. The liquid-absorbent sheets 11, 21, 31 may be formed of spun lace fibrous non-woven fabric made of hydrophilic fiber such as cotton having a basis weight in a range of about 14 to 40 g/m², preferably in a range of about 20 to 35 g/m², more preferably of about 30 g/m². The liquid-impervious sheets 12, 22, 32 may be formed of plastic film having a basis weight in a range of about 10 to 25 g/m², preferably of about 18 g/m². The liquid-impervious sheets 12, 22, 32 preferably have breathability.

The lower side, i.e., the side facing the wearer's garment of the third component pad 30 is formed with joining means 40 such as adhesive which is, in turn, covered from below with a releasable sheet 50. The releasable sheet 50 may be peeled off from the third pad 30 to expose the joining means 40 by the intermediary of which the third pad 30 may be fastened to the wearer's panty.

The first, second and third component pads 10, 20, 30 and the releasable sheet 50 are same in shape as well as in size one to another and respective have the dimension in the transverse direction X gradually reduced from longitudinally opposite ends toward a central region defined by the imaginary transverse center line Q-Q. By dimensioning the component pads 10, 20, 30 in this manner, it is assured that these component pads are put in close contact with the wearer's body without irregular deformation of these component pads in the crotch region.

The first, second and third component pads 10, 20, 30 are formed along the peripheral edge 2 thereof with individual temporarily joined regions 13, 23, 33. Specifically, these individual temporarily joined regions 13, 23, 33 comprise a plurality depressions (debossings) arranged intermittently along the peripheral edge 2. The depressions are formed by collectively hot-pressing these first, second and third component pads 10, 20, 30 in the thickness direction so as to define the continuous linear temporarily joined region along which these component pads may be temporarily joined together.

The temporally joints between the temporally joining regions 13, 23, 33 are easily peeled off by pulling upward the respective component pads 10, 20, 30 so as to be spaced from one another. Each of the temporarily joined regions 13, 23, 33 is interrupted along a partial segment 2A of the peripheral edge 2 and such segments 2A serve as guide regions to guide the wearer to the appropriate end regions of the respective component pads 10, 20, 30 to be pinched with the fingers when it is desired to peel off the respective component pads 10, 20, 30 from the composite pad 1 and thereby to facilitate the end regions of the respective component pads 10, 20, 30 to be pinched with the fingers.

The first, second and third component pads 10, 20, 30 as have been described above are respectively formed with identifying elements which are peculiar to the respective component pads 10, 20, 30. The identifying elements may be formed directly on upper surfaces of the liquid-absorbent sheets 11, 21, 31 of the respective component pads 10, 20, 30 or on the liquid-impervious sheets 2, 22, 32 so as to be visually recognized through the associated liquid-absorbent sheets 11, 21, 31.

### EXAMPLE 1

FIG. 3 illustrates identifying elements 61 through 63 used in Example 1 wherein graphics are used as the identifying elements. More specifically, FIG. 3 (a) illustrates the identifying elements 61 to be visually recognized in the first component pad 10, FIG. 3 (b) illustrates the identifying elements 62 to be visually recognized in the second component pad 20 and FIG. 3 (c) illustrates the identifying elements 63 to be visually recognized in the third component pad 30. On the first component pad 10, two groups of butterflies each consisting of three butterflies are drawn so as to be diagonally opposed to each other as identifying elements 61. On the second component pad 20, two groups each consisting of two butterflies, i.e., reduced by one from each of the groups on the first component pad 10, are drawn as identifying elements 62. On the third component pad 30, the total number of butterflies is further reduced to a single pair which is drawn as identifying elements 63. In this way, the wearer can easily know what number component pad is functioning or how many component pads are left to be used based on the number of butterflies drawn on the respective component pads as the identifying elements 61 through 63.

In this Example, if the last component pad, i.e., the third pad 30 is being used, a single pair of butterflies is displayed as the identifying elements 63 to indicate <this is the last component pad>. Even if it becomes unclear for the wearer what number component pad is being used, the wearer can understand that the last component pad is being used merely by counting the number of butterflies drawn on this component pad. Obviously the graphic used in the invention is not limited to the graphic of butterflies and it is possible to use the other various graphics such as snow crystal mark and heart mark.

As has been described above, even if it becomes unclear for the wearer how many component pads are left to be useful and/or what number component pad is being used, the wearer can understand it merely by counting the number of the graphics displayed on the current component pad. In this way, it is possible for the wearer to exchange the used composite pad with a fresh composite pad at the right time and to avoid wastefulness such that the used composite pad still containing many useful component pads might be thrown away.

### EXAMPLE 2

FIG. 4 is a view similar to FIG. 3, illustrating Example 2 wherein graphics are used as the identifying elements 64 through 66. The first component pad 10 includes a pair of green shoots drawn thereon as identifying elements 64, the second component pad 20 includes a pair of the green shoots grown-up to have blossom buds drawn thereon as the identifying elements 65 and the third component pad 30 includes a pair of the young shoots further grown-up to have flowers drawn thereon as identifying elements 66. These graphics represent a temporal change as a natural phenomenon. In general, a series of graphics which are suggestive of temporal change may be used as the identifying elements to assist the wearer to recognize what number component pad is being used.

Graphics being suggestive of a temporal change are generally meant by a series of graphics allowing the wearer viewing one of these graphics to forecast the following graphic and thereby to conceive a series of temporal changes. For example, as a typical temporal change presented by astronomical objects, a series of graphics representing the temporal change starting from full moon to half-moon and then to crescent moon can be used. It is also possible to use a series of graphics representing a temporal change such that the sun sets and the moon rises. It is also possible to use a series of graphics representing a temporal change such that an apple is munched and gradually gets chipped.

### EXAMPLE 3

FIG. 5 is a view similar to FIG. 3, illustrating identifying elements 67 through 69 used in Example 3. The first component pad 10 includes a numeral “3” displayed thereon as the identifying element 67, the second component pad 20 includes a numeral “2” displayed thereon as the identifying element 68 and the third component pad 30 includes a numeral “1” displayed thereon as the identifying element 69. In this manner, the remaining number of the component pads is directly displayed on the respective component pads and allow the wearer to recognize the number of remaining useful component pads. While the numeral “1” is displayed on the third component pad 30 to tell the wearer that this component pad 30 is a useful last component pad, it is also possible to display a numeral “0” on the third component pad 30 and thereby to suggest there is no more useful component pad.

These numerals may be replaced by letters having any concept of sequential as the identifying elements. “A”, “B”, “C” of the alphabet is a typical example of such letters having the concept of sequential.

In the respective Examples 1 through 3, the identifying element may be formed not only by printing or the like technique but also by locally embossing the surfaces of the respective component pads. In any case, it is essential that these identifying elements can be visually recognized from the side of the liquid-absorbent sheets 11, 21, 31 of the component pads 10, 20, 30.

While the respective Examples have been described on the assumption that each of them is the trilaminar composite pad, the invention is not limited to the trilaminar composite pad and the number of the component pads may be optional so far as the number of the component pads is two or more.

While the identifying elements 61 through 69 have been described to be displayed symmetrically about the imaginary longitudinal center line P-P as well as about the imaginary transverse center line Q-Q, the invention is not limited to this. For example, the identifying element(s) may be displayed at a single position. When the identifying element(s) is or are displayed only one of the opposite ends in the longitudinal direction Y, the wearer can recognize the orientation of the composite pad during its use on the basis of the position at which the identifying element(s) is or are displayed.

In the lowermost third component pad 30, fibrous non-woven fabric such as spun bond/melt blown/spun bond (SMS) non-woven fabric may be additionally laminated on the lower surface of the third component pad 30 and the joining means 40 is provided on this fibrous non-woven fabric. The non-woven fabric additionally laminated on the third component pad 30 serves as a reinforcing member preventing the third component pad 30 from being damaged in the course of peeling off the third component pad 30 from the wearer's garment such as panty for the purpose of exchanging the composite pad 1. By using the fibrous non-woven fabric as the reinforcing member, stiffness of the liquid-impervious sheet 32 constituting the third component pad 30 can be reduced and thereby feeling of discomfort which might otherwise be experienced by the wearer during use of the composite pad 1 can be alleviated.

1. A disposable bodily fluid handling composite pad having a longitudinal direction, a transverse direction, an upper side facing a wearer's body and a lower side opposite to said upper side, and comprising component pads each including an upper liquid-absorbent sheet and a lower liquid-impervious sheet wherein two or more of said component pads are laminated one on another, said disposable bodily fluid handling composite pad being characterized in that:
said two or more component pads respectively include temporarily joined regions along which said component pads are temporarily joined so as to be peeled off from the other component pads and identifying elements adapted to be visually recognized from said upper side; and
said identifying element displayed on one component pad is different from said identifying elements displayed on other component pads.

2. The disposable bodily fluid handling composite pad defined by claim 1, wherein said identifying element includes at least one of letter, graphic and symbol as a constituent.

3. The disposable bodily fluid handling composite pad defined by claim 2, wherein said identifying element includes a numeric conception as said constituent.

4. The disposable bodily fluid handling composite pad defined by claim 2, wherein said identifying element includes said constituent having a series of conception and displayed depending on order in which said component pads laminated one on another.

5. The disposable bodily fluid handling composite pad defined by claim 2, wherein said identifying element comprises the same number of said letter or graphic or symbol as the number of said component pads counted from said lower side.

6. The disposable bodily fluid handling composite pad defined by claim 2, wherein said identifying element comprises two or more of said graphics or symbols having a conception of temporal change and said graphics or symbols are differently displayed on each of the component pads so as to display said conception of temporal change corresponding to order of lamination of the component pads.

7. The disposable bodily fluid handling composite pad defined by claim 1, wherein said temporarily joined regions is formed along a peripheral edge of said component pads except a partial segment of the peripheral edge by a heat crimping treatment so that said partial segment left free from an effect of said heat crimping treatment serves as a guide region to indicate an appropriate region to be pinched by the wearer's fingers when it is desired to be peeled off the soiled component pad from said composite pad.
